(19) United States
(12) Patent Application Publication
(10) Pub. No.: US 2020/0038542 A1
(43) Pub. Date: Feb. 6, 2020

(54) Method for economically disinfecting air using light fixtures

(71) Applicants: Philip George Franklin, Santa Ana, CA (US); Jeffery Jay Collins, Irvine, CA (US)

(72) Inventors: Philip George Franklin, Santa Ana, CA (US); Jeffery Jay Collins, Irvine, CA (US)

(73) Assignee: Skizukesa Technologies LLC, Santa Ana, CA (US)

(21) Appl. No.: 15/998,322

(22) Filed: Aug. 6, 2018

(51) Int. Cl. A61L 9/20

(52) U.S. Cl. CPC A61L 9/20; A61L 2209/12; A61L 2209/212; A61L 2209/16; A61L 2209/14

(57) ABSTRACT

A method is revealed that teaches that a UV or near-UV lighting source inside a lighting fixture can be used to fulfil two functions; (1) First, to disinfect room air of bioaerosols and remove certain air-borne chemicals; and then (2) To excite white or other color phosphor compounds, so that the final output of the lighting fixture is that of visible non-UV light. The method results in a minimal increase in operating energy costs over that of a standard LED light fixture, but with the added benefit of providing air disinfection and purification.

### TECHNICAL FIELD

The present invention teaches a method of using a modified lighting source, wherein the light fixtures can be used to disinfect the building's atmosphere of harmful bacteria, viruses, molds, mildews, and protozoa, linked to airborne acquired infections, while not significantly increasing energy costs as other methods require.

### CROSS REFERENCE TO RELATED APPLICATIONS

None.

### BACKGROUND AND DISCUSSION OF PRIOR ART

Throughout the world there are buildings where people concentrate during the day, at night, or both day and night. Because of the functions of some of certain buildings, like hospitals and doctor's offices, the proportion and number of persons (vectors) carrying harmful infections is increased. Additionally, there are some places within certain facilities, such as school and university hallways, that are at times packed with persons at levels to near standing room only capacities, providing ideal conditions for the transfer of airborne diseases to a multiplicity of persons at a time.

Hospital Acquired Infections (HAI's), especially those caused by Staphylococcal Aureus and Clostridium Difficile, cost hospitals an extraordinary amount of money every year to try to prevent transmission within a facility. If a transference to a patent does occur, the hospital has the further expense to cure the infection as it was likely Hospital Acquired.

Simple statistics reveals that crowded hallways of academia facilities results in waves of disease and infections (influenza for example) spreading faster and with greater ease then that of less crowded facilities and areas. Indeed, subjectively speaking, families often speak of children bringing disease, such as colds and the flu, home to their parents and siblings as the sequence of events of the spread of disease from school to the family.

In agribusiness, there is a need to grow certain food or plant crops indoors in an artificial environment wherein the plants must avoid exposure to bioaerosols. As an example, but not as a means of limitation, compliance with recent governmental rules and regulations for the quality and purity of marijuana growth and cultivation would be greatly aided by the method revealed herein.

There are methods of limiting the airborne spread of viruses and bacteria in buildings such as:
a. The application of liquid applied disinfectants by staff to area floors and surfaces;
b. Mass air-sprayed disinfectants (using a machine that creates a fog of chemical disinfectant that fills a room, applied while no one is in the room or area);
c. The use of High Efficiency Particulate Air (HEPA) filters in room air systems, sometimes used in conjunction with UV-C;
d. The production and release of Ozone into HVAC (Heating, Ventalation, and Air Conditioning) systems to oxidize air;
e. Saturated Ultra-Violet illumination and disinfection (with no one in the room or area for fears of causing skin cancer or optical eye injury by exposure to the UV);
f. Limited UV-C illumination directed and focused to room ceiling areas only, and away from human occupancy; and
g. Area illumination by near UV (405 nm) blue light (which humans can be safely exposed to).

The art that is revealed by the invention is in room air disinfection of bioaerosols. Bioaerosols are natural or artificial particles of biological (microbial, plant, or animal) origin suspended in the air. Bioaerosols may consist of bacteria, fungi, spores of fungi or viruses, viruses, microbial toxins, pollen, plant fibers, etc. (Douwes, J., et al., Bioaerosol health effects and exposure assessment: Progress and prospects. Annals of Occupational Hygiene, 2003. 47(3): p. 187-200.)

When it comes to disinfecting room air, HEPA filter techniques—especially combined with a UV disinfection light embedded in the filter housing—are effective but often prohibitively expensive due to filter cost, filter size and complexity, the need for such filters to be installed in series with HVAC ducts, electrical operational cost of UV-C light source, and the labor costs needed to replace the filters often.

Area illumination by near UV (405 nm) blue light is a good solution for disinfection of room surfaces over time. For example, in U.S. Pat. No. 9,039,966B2, “Inactivation of gram positive bacteria”, to University of Strathclyde, 2006 Jul. 28, they teach light fixtures that facilitate co-generating near-UV blue light and white light out of light fixtures at the same time. The blue light output is necessarily restricted so that occupiers of the room perceive white light as being the predominate light. However, such disinfection, because of limited intensity of blue light reaching the surface areas of the room, is limited to the disinfection of only certain bacterial species, and then only after a relatively long exposure (too long of an exposure necessary to disinfect moving air). Additionally, the effectiveness of said method inactivating bioaerosols is further limited as the near-UV blue light at 405 nanometers is outside of the UV-C band of light that is most effective at disinfecting bioaerosols.

As a further limitation of U.S. Pat. No. 9,039,966B2, “Inactivation of gram positive bacteria”, to University of Strathclyde, 2006 Jul. 28, the use of near UV blue light if operated in mixed mode with white light almost doubles the needed electrical energy to operate the light fixture, thus negating some of the energy-savings otherwise realized by upgrading to LED lighting. Additionally, the manufacturers of such blue light systems also suggest that room occupancy sensors be install so that the blue light output can be intensified when humans are not in the room (because the ambient color of the room will no longer be white, but shifted to blue), thus adding to the further cost of efficiently employing this method.

In Patent US20090041632A1, “Air Purifier System and Method”, to Edwin David Day, John Douglas Pink, and Robert Brian Knight, 2009 Feb. 12, using UV-C in combination with an air filter, and adding a chemical-based process, is revealed. However, the inventors fail to realize as our method does, that UV-C energy need not be discarded, but can be used to generate white light. Hence, in this patent the UV-C energy—and the electrical energy used to generate it—is in effect thrown away and wasted.

In U.S. Pat. No. 6,022,511A, “Apparatus and Method for Germicidally Cleaning Air in a Duct System” to Arthur L. Matschke, 1998 Jul. 9′, disclosed is a device that can be added to new or existing HVAC duct work that uses UV-C light to perform air disinfection. However, disinfection by UV—C is all that is taught, and again Matschke fails to realize that UV-C energy can be used to generate white light. Hence, yet again, in this patent the UV-C energy—and the electrical energy used to generate it—is in effect thrown away and wasted after it is used to disinfect light. In the case of exposed duct work in industrial applications white light generation from his method could possibly be used in lieu of a light fixture, thus reclaiming the energy that is discarded.

Patent US20060008391A1, “Nano Electro-Optical Air Sterilizer and Fresh Air Maker with Ionizer”, to Se Yuen, 2004 Jul. 12, teaches the design of a portable air filtration device, but like the previous examples, again fails to realize that the UV-C energy, instead of wasted, can be used to generate white light.

In a similar fashion, U.S. Pat. No. 3,967,927, “Decorative ultraviolet lamp fixture”, to Lawrence Patterson, 1976 Jul. 6; U.S. Pat. No. 5,833,740A, “Air Purifier”, to Normand Brais, 1996 Nov. 25; U.S. Pat. No. 6,818,177B1, “Ultraviolet Air Purification Systems”, to Mark Turcotte, 2004 Nov. 16; and U.S. Pat. No. 7,326,387B2, “Air Decontamination Devices”, to Theodore A. M Arts, James M Thomsen, Paul J Chirayath, Jerome Schentag, 2008 Feb. 5″; all teach the disinfecting and germicidal benefits of UV-C irradiation of air, but all discard the UV-C energy at the end of their process, instead of realizing the UV-C can be used to perform one more function: Generate white light useful for illuminating building spaces, thus somewhat recouping the cost of the electrical energy used to power the device.

The maximum absorption of UV light by the nucleic acid, DNA′ and RNA, occurs at a wavelength of 260 nm. Because of the physics involved, both fluorescent and LED UV-C lamps are closest to 260 nm at an output of 254 nm. The germicidal lamp emitting UV at 254 nm is operating very close to the optimized wavelength for maximum absorption by nucleic acids.

Laboratory testing confirms that UV sources operating in the range of 250 nm to 260 nm (designated as a sub-set of “UV-C” wavelengths) are the most efficient at disinfecting the targeted microorganisms, with very little to no differences between operation at 260 nm verses 254 nm. Such wavelengths of UV light can damage human tissues if such tissues were to be exposed to significant intensities of the light. This is especially true of skin tissue (resulting in melanoma skin cancer) and the tissues of the human eye (resulting in direct and indirect damage to eyesight that may be temporary or permanent).

What is needed is a method that will disinfect room air, without requiring large and bulky HEPA air filters, employs the more efficient 254 nm UV-C light without chance exposure to humans or animals, without the need of application only if the room is empty, and does not result in a significant increase in electrical draw or consumption.

### BRIEF SUMMARY OF THE INVENTION OF THE INVENTION

The invention teaches a method wherein atmospheric air can be disinfected using UV or near-UV light, and then that same light source is then used for generating a second light source: That of white or other colors of visible light for use for general lighting. The method realizes power savings compared to other solutions that need to power separate UV and white lighting photonic sources.

### MORE-DETAILED SUMMARY OF THE INVENTION

To be clear, the method that is invented is that a UV or near-UV light source can be used to do two things in sequence:
a) Disinfect and purify air, and instead of, as other methods do, discarding the UV or Near-UV photonic rays, the invention teaches that the UV energy can then:
b) Be used to produce visible light, generally, though not exclusively, through the excitation of phosphors, thus “not wasting” the UV or Near-UV energy otherwise discarded.

The first embodiment of the invention teaches a method wherein a white light fixture used for area illumination, can generate its white light output by utilizing an internal UV light source, which is ultimately applied to a white phosphor coating in or on a lens; Before application to the white phosphor, however, said generated LW light is used to disinfect and clean atmospheric air of bioaerosols and certain air-borne chemicals as it passes through the UV light.

Thus, the method teaches that without the need of installing and powering separate white and UV or near-UV light sources, air can be disinfected using UV light from the same photonic source that generates the photons used for generating the white light, thus realizing power and component savings compared to other solutions by eliminating the need to power separate UV and white lighting photonic sources. Additionally, the method provides additional control device savings, and enhanced safety for occupants, as UV light never emanates from the light fixture.

That is, the invention teaches a method for using a single electrically-powered light source to generate UV Photonic energy that is used to disinfect air of bacterial, viruses, mold, and other bioaerosols; and then use that same UV photonic energy left over after air disinfection to bombard and excite white phosphors to generate white light useful for general illumination.

In general terms, most LED semiconductors operate on the basis of first causing an electron within an atom to jump from a ground state orbital to a higher-energy orbital, and then when the electron jumps back to the ground state, a photon is emitted. The emitted photon is at a single dominate wavelength (color) of light, the wavelength of which is dependent upon the distance of the jump from the higher orbital back to the ground state. Thus, LED semiconductors predominately output only a single wavelength (color) of light.

The result is, white light is difficult to generate out of basic LEDs because white light is composed of a spectrum of colors, and not just a single wavelength of light. It is often said that black can be thought of as an absence of all colors, while white can be thought of as the presence of all colors.

White light is a mixture of the primary or secondary optical colors, therefore single-die LED semiconductors cannot be used to directly emit white light, as their photons are limited to one or a narrow-range of wavelengths. However, to manufacture a white-light emitting LED assembly (lamp), there are two solutions to this limitation, which industry commonly uses.

In one common solution, white light can be generated by employing three or more different LED semiconductor chips or dies within a single LED lamp to emit the primary colors Red, Green, and Blue (“RGB”). Such LEDs have the primary light outputs set so that the outputted white light has a fixed color temperature in the white-light Kelvin range (“Warm White” verses “Cool White” verses “Daylight White” LED Lamps etc.). However, this three-color technique is expensive, complicated, and often results in color-temperature variations that are costly to control during the manufacturing process. Therefore, because of the expense of manufacturing an LED Lamp of this sort, this color-mixing technique is typically used when another factor, other than price, dominates the application (such as the need for high color rendering which the next technique used to lack before phosphor improvements).

The second technique mirrors the operation of a fluorescent lamp. In fluorescent lamps, electric currents are used to generate an arc inside a tube filled with mercury vapor. The arc ionizes the mercury mixture and upper-spectrum UV light is emitted by the ionized gas. The UV light in turn is used to excite molecules within a white phosphor coating added to the inside of the glass tube to realize the emitting of white light out of the lamp.

White-light emitting LED Lamps can employ a similar technique as used in the operation of fluorescent lamps: Electrical energy is used to generate UV light first, and then that UV light is used to excite a white phosphor coating which in-turn generates white light. In such an LED Lamp, electric currents in a semiconductor chip are used to generate upper-spectrum blue or UV light, which in turn is used to excite molecules within a white phosphor coating applied over or near the semiconductor chip. Thus, a single wavelength of UV light emitted from a semiconductor, is applied to and excites white phosphors, which in-turn emit the desired multi-spectrum white light.

The method teaches that UV photonic energy can be used to first disinfect air, and then be applied to excite the white phosphors. In the first embodiment of the invention this is done by using an array of UV LED Chips or lamps to create a significant source of UV photonic light, and then installing a white phosphor film at a distance from the UV light source, creating an air gap between the UV source and white phosphor film. Air that is to be disinfected is passed through the air gap between the UV source and the white phosphor assembly, thereby facilitating bioaerosols being exposed to high-intensity UV photonic radiation.

With exposure to sufficient UV photonic energy (“UV Radiation”), bioaerosols are rapidly and effectively rendered incapable of reproduction or infection through a physical rather than chemical process. As air is passed between the gap of the UV source and white phosphor film, the bacteria, viruses, and other bioaerosols are bombarded with high-energy UV photon emissions that strike the bioaerosols, and create an internal process whereby the bioaerosol is immediately inactivated and later dies.

Microorganisms, such as bioaerosols, are inactivated by UV light through a process that results in photochemical damage to the nucleic acids within the microorganism. The high energy photons of UV light are absorbed by cellular DNA and RNA. Research has shown that UV light centered around 254 nanometers is at the peak of germicidal effectiveness against most microorganisms. Laboratory experiments demonstrate that the absorption of the high-energy UV photons forms new covalent bonds between adjacent nucleotides within the microorganism's DNA and RNA, and this ultimately results in the formation of thymine dimers—damage to the DNA or RNA genetic sequence—which prevent replication of the microorganism and fully block the ability to infect other organisms. The organism dies because the formation of such covalent bonds prevents the DNA from being unzipped for replication by RNA, and therefore the organism is unable to reproduce. When the organism does try to replicate, it starts to unzip the DNA, and then dies midway through the process when it is blocked from unzipping the DNA further.

UV light is comprised of the most energetic photons within the light spectrum. Studies have shown that light emissions in the range of wavelengths between 200-300 nanometers are especially germicidal.

Field and laboratory testing has demonstrated that a UV dose of 12 mJ/cm² at 250-260 nanometers is sufficient to create enough photochemical damage to render bioaerosols safe, including rendering them unable to repair themselves.

An added benefit to using the method revealed herein is that many airborne industrial solvents and pesticides, are neutralized through an oxidative process when bombarded by High-Energy UV-C light.

Since there is no need for the use of chemicals for disinfection, UV light has been enjoying widespread adoption as a safe and environmentally-friendly method for disinfection of both air and water.

In humans, the damage to DNA and RNA from thymine dimmers can result in skin cancer (melanoma). Furthermore, the oxidative photochemical damage that results from UV exposure can result in damage to vision (e.g. UV-related keratoconjunctivitis). For these reasons and others, humans and animals should not be exposed to the concentrated UV light that is under discussion. Indeed, from a purely public relations standpoint alone, the light fixtures that employ the revealed method should be so designed as to prevent any escape of the UV spectrum from the fixture, and be compliant to such standards as International Commission on Non-Ionizing Radiation Protection—Guidelines on limits of exposure to ultraviolet radiation of wavelengths between 180 nm and 400 nm (incoherent radiation) Health Physics 87, 171-186; 2004, and International Commission on Non-Ionizing Radiation Protection—Guidelines on limits of exposure to optical radiation from 0.38 to 3.9 mm. Health Physics 73; 539-555; 1997.

Thus, by employing the method revealed by the herein and the embodiments, variations, and aspects of the invention, lighting fixtures commonly within buildings can be used to efficiently and promptly disinfect air and provide general lighting, without a significant increase in electrical energy demands.

### Advantages

The advantages of the method revealed include, but are not limited to:
Cost Advantage: The electrical energy required is commensurate with a typical LED fixture of the same size and lumen optical output. No significant additional load of the electrical system is required to operate additional LEDs or disinfection sources;
Cost Advantage: No need to hide or install bulky and expensive HEPA filters;
Cost Advantage: No need to monitor room occupancy to protect humans and animals from UV exposure;
Safety Advantage: UV light is not emitted outside of the light fixture;
Safety Advantage: Effective against a broad range of microorganisms;
Safety Advantage: Disinfection by UV—C is a chemical-free process that adds nothing to the air or environment except UV-C light. No applications of chemicals are needed to effect or aid in disinfection;
Safety Advantage: UV-C treatment does not require transportation or storage of corrosive or toxic chemicals, as certain other methods require;
Safety Advantage: UV-C treatment does not create any carcinogen by-products that could adversely affect air quality or long-term human health;
Safety Advantage: Decontamination via oxygenation of several airborne solvents and pesticides;
Safety Advantage: Decontamination of many agents used as biological weapons;
Cost Advantage: Although possible, there is no need to plumb HVAC air flows to the lighting fixtures;
Cost Advantage: Periodic cleaning and electrical consumption comprise the only operating costs; and
Cost Advantage: Costs for leak response, employee training, employee safety gear, risk management, and emergency planning, are eliminated compared to chemical disinfection solutions.

### BRIEF DESCRIPTION OF THE DRAWINGS

FIG. 1 is a graphical representation of a typical white-light emitting LED Lamp that is based upon white phosphor excitation. The graphical representation is simplified so the discussion of the very basis of operation of the method can be demonstrated;

FIG. 2 is a minimal graphical representation of an array of UV LEDs being used to excite a White Phosphor Screen. These two sub-assemblies—UV Light Source Array and White Phosphor Screen—form the basis for the light-engine for a light fixture that illustrates the first embodiment of the invention;

FIG. 3 is a more complete graphical representation of the basis of a light-engine for use in the first embodiment of the invention. The light-engine consists of the UV LED array, and the phosphor film assembly of FIG. 2, enclosed in an air channel;

FIG. 4 is a graphical representation of the light-engine assembly of FIG. 3 as seen from an alternate overhead view;

FIG. 5 is a graphical representation of two light engines installed into a light fixture, wherein fans are used to supply air to the light engines;

FIG. 6 is a block diagram representation of a power supply capable of operating a light engine and fan;

FIG. 7 is a graphical representation of an alternative configuration of a light engine employing the method;

FIG. 8 is a graphical representation of another alternative configuration of a light engine employing the method;

FIG. 9 is a graphical representation of an alternative configuration of a light fixture wherein the light fixture is ducted to the HVAC system.

### LIST OF REFERENCED LETTERS AND NUMERALS

A Air Channel
L Light Engine
100 LED Lamp Wire Lead
110 LED Lamp Semiconductor Die
120 UV Light Rays
130 White Phosphor Coating
140 LED Lamp Molded Housing
150 LED Lamp Lens
160 White Light Rays
170 LED Lamp
200 Printed Circuit Board
210 LED Lamps
220 UV Light Rays
230 White Phosphor Target
240 White Light Rays
300 Printed Circuit Board
310 Flat LED Light Chips
320 UV Light Rays
330 White Phosphor Coating
340 Light Engine Enclosure
350 White Light Rays
400 Incoming Air End of Enclosure
410 Exiting Air End of Enclosure
500 Lighting Fixture Enclosure
510 Lighting Fixture Mounting Frame
520 Raised Cover Assembly
530 Side-Mounted Electrical Conduit Entry
540 Fan
550 Light Engine Main Body
560 Exiting Air Vent
570 Power Supply
580 Power Supply
600 Building Power Connector #1
610 Building Power Connector #2
620 Rectifier and Filter Circuitry
630 Switched-Mode Power Supply Circuitry
640 Fan Power Fuse and Connector Circuitry
650 Current Limited LED Circuitry and Connector #1
660 Current Limited LED Circuitry and Connector #2
670 Current Limited LED Circuitry and Connector #3
680 Current Limited LED Circuitry and Connector #4
700 Enclosure
710 Printed Circuit Board
720 LED Lamp
730 Encapsulation Material
740 LED Lamp Lens
750 UV Light Rays
760 White Light Rays
800 Printed Circuit Board
810 LED Lamp
820 UV Light Rays
830 Enclosure
840 Light Housing Sheet Metal
850 White Light Rays
900 Light Fixture Housing
910 Light Fixture Mounting Frame
920 Raised Cover Assembly
930 Side-Mounted Electrical Conduit Entry
940 Exiting Air Vent
950 Light Engine Assembly
960 Power Supply
970 Power Supply
980 HVAC Duct Connection

### Detailed Description—First Embodiment

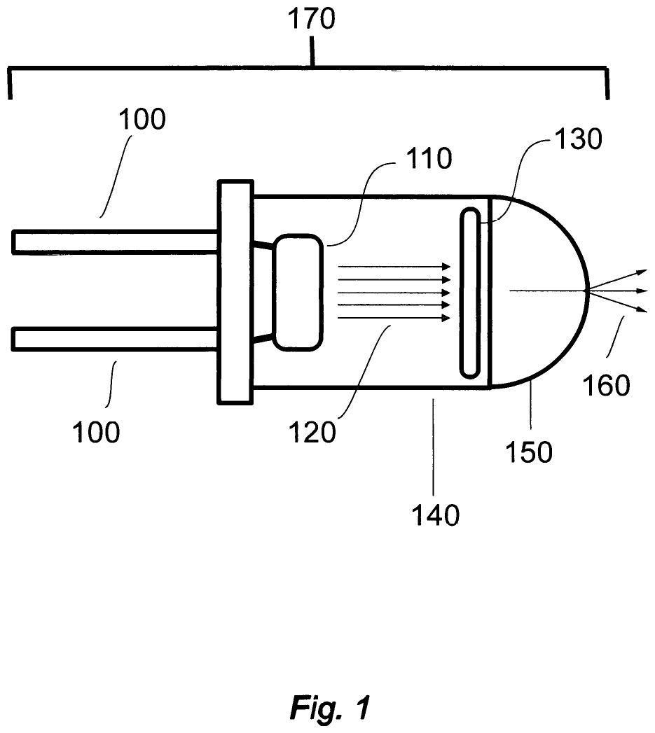

### FIG. 1—LED Lamp as Prior Art

FIG. 1 is included as an aid and example of prior art and background. FIG. 1 is a graphical representation of a typical 5 mm white-light emitting LED Lamp that is based upon white phosphor excitation. The graphical representation is overly simplified on purpose in that white phosphor coatings normally cover the entire LED die, and is not a simple target as shown here. While FIG. 1 is based upon a common and typical 5 mm LED Lamp, since dimensions are not shown as the representation is not intended to limit the method or application to any particular size or shape of LED Lamp. Indeed, the LED Lamp could be not as illustrated, but just a semiconductor chip with a white phosphor coating applied over said chip.

FIG. 1 depicts an LED Lamp (170) that is in the shape and form of a common 5 mm “T-1¾” LED Lamp, encased in a clear plastic casting (140), said casting employed to hold and protect the lamp's elements from movement or damage from environmental exposure.

In FIG. 1, (100) are conductors used to supply electrical energy to the LED Semiconductor Die (110) located within the plastic casting (140). In practice, if the LED Lamp were designed to operate on Direct Current, the conductors (100) would be polarized so that a positive voltage would be applied to one conductor, and a more negative voltage applied to the other. If the LED semiconductor die were designed with internal rectifiers, and hence designed to operate on Alternating Current, the conductors (100) would not be polarized. For purposes of simplification, the conductors (100) are shown without reference to polarity.

In this discussion the LED Semiconductor Die (110) emits ultra-violet light at or near a UV-C wavelength of 254 nanometers. The emitted UV rays (120) are projected towards a film of white phosphor (130). The white phosphor can be any of Y₃Al₅O₁₂:Ce³⁺ phosphor (white emission with a broad yellow band), Sr₂Si₅N₈:Ce³⁺ phosphor (white emission with a broad yellow band), M₂Si₅N₆:Eu²⁺ (where M=Ca, Sr, or Ba—white emission with a broad orange-red band), or any of the other phosphors used in industry.

The rays of white light (160) emitted from the film of white phosphor (130) are focused by the lens (150) and thereafter emitted out the lamp (170).

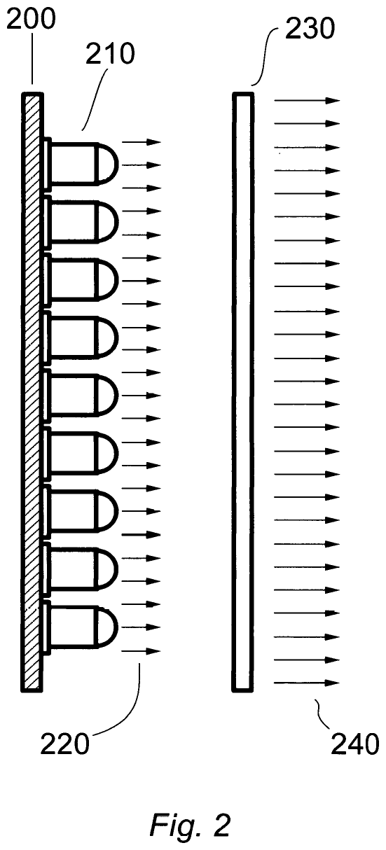

### FIG. 2—Pertinent Sub-Assemblies and the Basic Concept of the Method

FIG. 2 is a graphical representation of the two active sub-assemblies that together for the basis for a light engine that employs the method of the invention. The first active sub-assembly consists of a printed circuit board (200) to which a multiplicity of UV-C LED Lamps (210) are fitted to. The PCB (200) contains the circuit traces necessary to properly supply power to the Lamps (210). The PCB (200) and LED Lamp (210) subassembly is an array of rows and columns of LED Lamps. Only the first column of LED Lamps is visible in this graphical representation. The LED Lamps emit rays of UV-C light (220) through the air gap, and on towards the white-phosphor target.

The second active subassembly is the white-phosphor target, which is a coating, film, or sheet, of white phosphor applied to clear plastic or glass (230). When the rays of UV-C light (220) strike the phosphor target (230), molecules in the white phosphors excite, and emit rays of white light (240).

Together the two subassemblies first generate the UV-C light, and then convert the UV-C energy to a spectrum of white light, through the excitation of white phosphors. The air gap between the two active subassemblies of the light engine, is dependent on design, including air flow requirements, and needed UV-C dosing.

UV-C dosing is dependent upon air flow rate, and luminance of the UV-C reaching the microorganisms. One formula for determining dosing is “μWs/cm²=UV-C Intensity in μW/cm² times Exposure in seconds”.

Microorganism exposure time to the UV-C is directly to related to the length of the air channel and hence directly related to the length of the light engine. Exposure time is also inversely related to the travel speed of the air moving through the light engine.

Applying the formula to even the shortest light engine (as one in a simple two-foot long lighting fixture), and with the maximum air flow rate, we find that the total dose—given the intense UV-C generation by the LED array in close proximity to the air chamber—easily exceeds (by several times) the published standard of a UV dose of 12 mJ/cm² at 250-260 nanometers (a dose sufficient to create enough photochemical damage to render bioaerosols safe, and even render them unable to repair themselves).

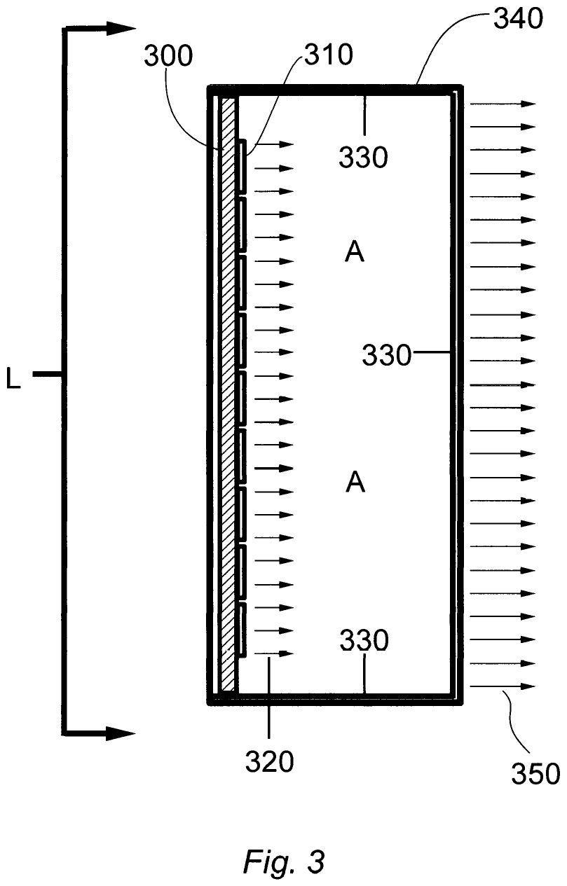

### FIG. 3—Introducing the Light Engine of the First Embodiment

FIG. 3 is a graphical representation of a side-view of a Light Engine (L) suitable for the First Embodiment of the invention. The Light Engine assembly (L) is the active light-generating subassembly within a light fixture that uses the revealed method, and can be thought to be analogous to a fluorescent lamp in a fluorescent light fixture.

In FIG. 3, (300) is a Printed Circuit Board (PCB) that the UV-C LEDs (310) and associated control circuitry is attached to. In this embodiment, the LEDs (310) employed are flat dies or wafers, so as to minimize dust accumulation. The PCB (300) and LEDs (310) can and likely will be, encapsulated with a coating (not illustrated in the thawing) such that the PCB (300) and LED (310) components as an assembly will be protected from environment damage such as water damage, and aid in the ease of dusting or cleaning of the air channel (A) of the light engine. The PCB (300) including LEDs (310) is fixed to one of the long sides of transparent enclosure (340).

In the first embodiment, the enclosure (340) is a rectangular extrusion made from a transparent material, such as glass or plastic. The center of said transparent enclosure (340) creates the air channel (A) within the Light Engine (L), wherein the air that is to be exposed to the high-intensity UV-C light will travel. As a minimum, the remaining three sides of enclosure (340) have applied to them a coating of a suitable white phosphor (330) applied inside the enclosure (340). It is anticipated that all sides of the inside of enclosure (340) may be coated with white phosphor (330), or as another variant, the white phosphor (330) may be coated to the outside of enclosure (340).

Further still, it is anticipated that the white phosphor compound (330) may be incorporated into the material that makes enclosure (340). E.g. A white phosphor compound (330) may be added to a plastic compound, from which enclosure (340) is later extruded, thus negating the need to apply a separate white phosphor coating (330) to the enclosure (340).

The UV-C rays (320) are concentrated within the air channel (A) of the enclosure (340), and thus bioaerosols and other compounds are rapidly disinfected or neutralized as they pass through the length of the Light Engine (L). The UV-C light rays (320) strike the applied white phosphor coating (330) and white light rays (350) emanate out of the Light Engine (L).

Air applied to the Light Engine (L), travels through the Light Engine in the air channel (A) created by enclosure (340). The air may be supplied via a connection to the HVAC system through the light fixture, or the light fixture may supply air via a separate fan assembly, or thermal currents may be created in the light fixture to move the air along inside the Light Engine (L).

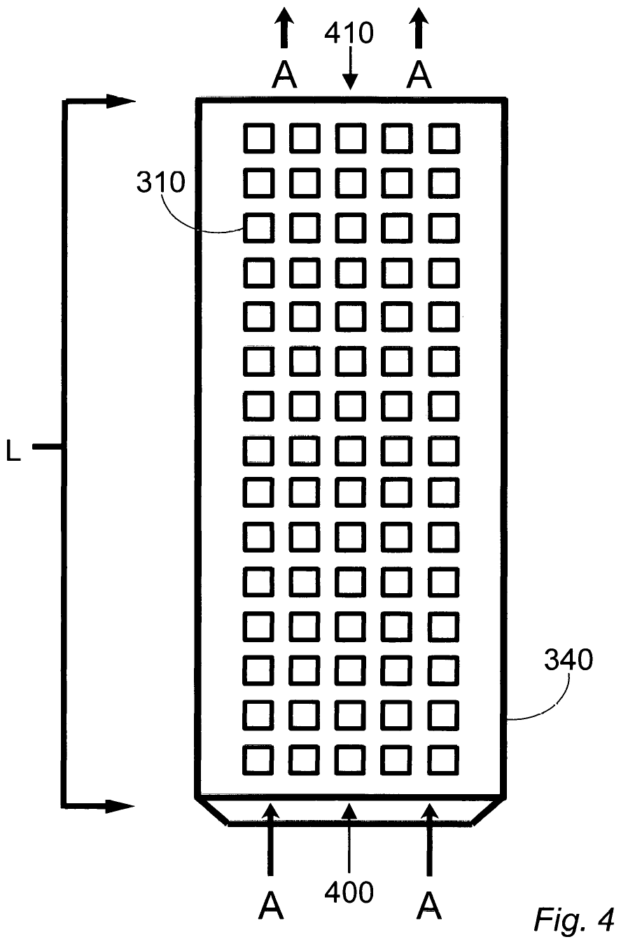

### FIG. 4—the Light Engine of the First Embodiment Viewed from the Top

FIG. 4 is a graphical representation of an alternative “top” view of the same Light Engine (L) as depicted in FIG. 3. The Light Engine is shown here as being relatively short in length. In practice the Light Engine (L) and hence its enclosure (340) would often be just under two feet in length, and may be just under four feet in length depending on the needs of the light fixture housing. Also, dependent on the light fixture housing, there may be two or more Light Engines installed in the fixture.

The LEDs (310) are shown as if the enclosure (340) were transparent. In practice the white phosphor coating or compound would prevent the LEDs being visible. Indeed, said white phosphor is necessary to prevent UV-C energy release from the Light Engine (L) to the outside environment.

In the case of this illustration, air flow (A) is depicted traveling up the drawing. That is, air (A) enters at the bottom opening (400) of the enclosure (340), and exits from the top opening (410). The angled view (400) of the opening of the enclosure (340) helps to illustrate that the enclosure (340) is rectangular.

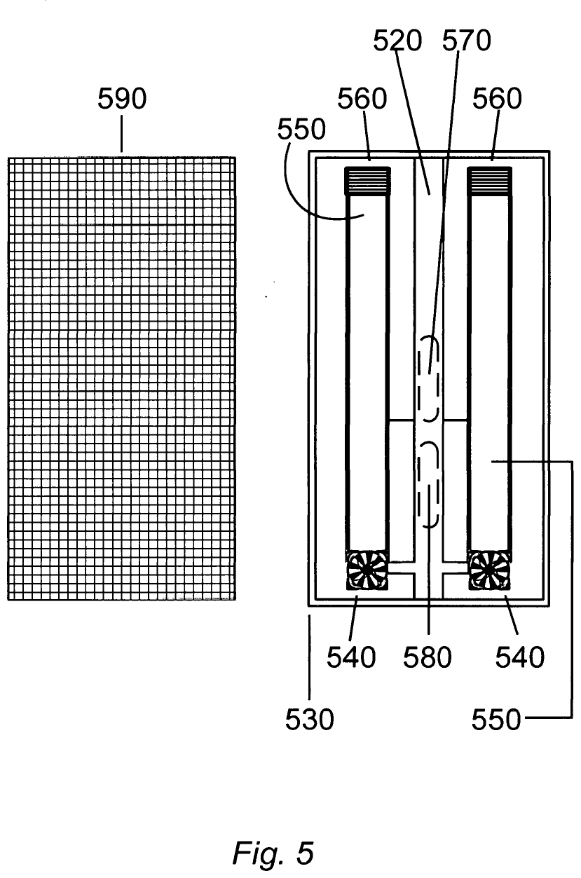

### FIG. 5—an Example of a Light Fixture that Employs the Method

FIG. 5 is a graphic depicting a simple 2-foot by 4-foot light fixture (500) that is in keeping with the spirit of the first embodiment. The light fixture (500) holds two Light Engine Assemblies and has a frame (510) around the edges of the light fixture such that the fixture can be hung in a typical 2-foot by 4-foot gridded drop ceiling.

The Light Engines (550) are fitted with air fans (540), and output air vents (560). In this embodiment, the fans (540) create a positive pressure, forcing untreated air into the air channel of the light Engines (550). The treated air exits the Light Engines (550) via vents (560) installed at the exit of the Light Engines (550). A Light Engine (550), coupled to a Fan (540), and coupled to an Exit Vent (560), together creates a Local-Fan Light Engine Assembly. It is anticipated that the fans (540) may also be fitted with a simple air filter to help reduce dust deposits in the Light Engine (550). It is also anticipated that the fans (540) may have a decorative plate that hides them from view from anyone standing under the fixture.

Electrical power can enter and exit the fixture (500) via side-mounted conduit connectors (530) located at both ends of the fixture. Raised cover assembly (520) facilitates locating the electrical power supplies (570) and (580) needed to support the fixture, as well hiding and protecting electrical wiring. Pathways from the raised cover facilitate electrical connects to each Local-Fan Light Engine Assembly [(540), (550), and (560), taken together as an assembly].

The power supply circuitry (570) and (580) are shown in dashed lines as they are located behind the raised cover (520), and therefore not normally visible.

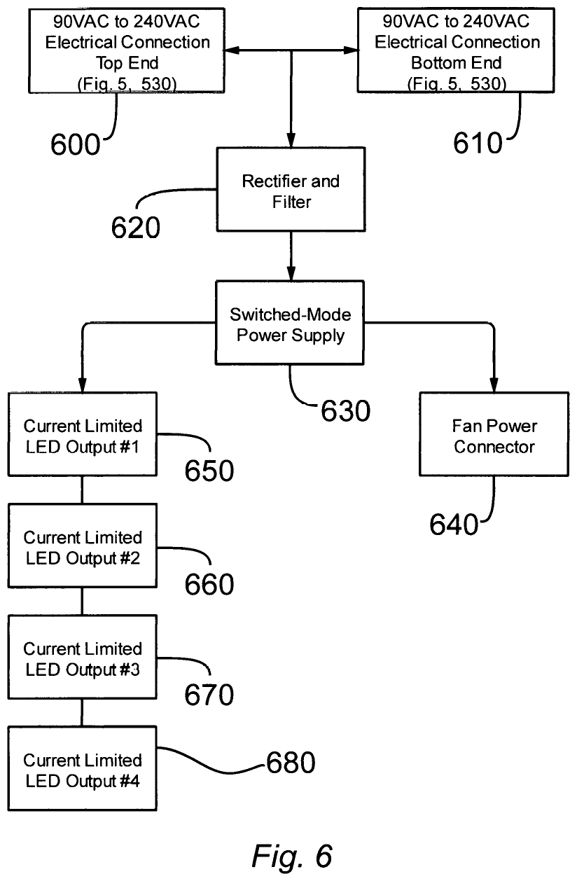

### FIG. 6—Power Supply Block Diagram

FIG. 6 is a block diagram of the power supply circuitry (FIG. 5, 570) and (FIG. 5, 580) used to power the Local-Fan Light Engine Assemblies. The block diagram is for power circuitry needed to run one Local-Fan Light Engine Assembly. Therefore, a dual Light Engine lighting fixture as depicted in FIG. 5 will require two power supplies.

In FIG. 6, (600) and (610) represent the connections to building power, or the internal connections needed to wire multiple power supplies inside the lighting fixture. The building power input is universal in that any voltage between 90 VAC and 240 VAC will operate the power supply circuitry. In this embodiment, (600) and (610) are wired directly to each other, and therefore can facilitate easy series wiring of the power connections.

The building power coming in from connections (600) and (610) are fused, rectified, and filtered in block (620). A high-voltage filtered direct current comes out of the Rectifier and Filter block (620) and is fed to the Switch-Mode Power Supply circuitry block (630) which chops, regulates, and filters, the high-voltage DC to lower voltages usable by the Light Engine Fan and LEDs.

The Fan Power Connector block (640) interfaces 12 VDC from the Switch-Mode Power Supply to the fan of the Local-Fan Light Engine via fuse and over-voltage protection devices.

There are four Current Limited LED Outputs (650), (660), (670), and (680), all powered by voltage and current from the Switch-Mode Power Supply. Current-Limited LED Output #1 (650) provides fusing and overvoltage protection, and includes circuitry that limits the current that powers a string of LEDs, ensuring that each LED runs at the same brightness, and protects other LEDs in the string should one or more LEDs short-out.

Current-Limited LED Output #2 (660), and Output #3 (670), and Output #4 (680), perform the same functions as Current-Limited LED Output #1 (650). Each output [(650), (660), (670), and (680)] powers one-quarter of the LED array, assuring continued operation of three-quarters of the LEDs should one output fail.

This circuitry and all the support wiring is mounted to the light fixture housing (FIG. 5, 500), and located under the raised cover (FIG. 5, 520).

### Operation—First Embodiment

The light fixture (FIG. 5, 500) is mounted to a 2-foot by 4-foot grid opening in a drop ceiling. The raised cover piece (FIG. 5, 520) is removed, providing access to the lighting fixture connections, so building power connections can be made. The building electrical power is brought to the electrical entry holes (FIG. 5, 530), and connection is made to the fixture's electrical connectors on the power supply boards. The raised cover (FIG. 5, 520) is replaced in the fixture.

When powered up, the power supplies (FIG. 5, 570) and (FIG. 5, 580) in the fixture will supply the outputs necessary to run the Local-Fan Light Engine Assemblies.

The fans (FIG. 5, 540) will force untreated air into the light engine's air chambers (FIG. 4, 400) and the air will be disinfected by the UV-C light emanating from the multiplicity of LEDs. The treated air will then exit the air chamber via the exit vents (FIG. 5, 560).

The UV-C light, after treating the air, will excite the white phosphor of the Light Engine (FIG. 3, 330), and the exiting light will be focused down from the ceiling to cover the building area below the lighting fixture (FIG. 5, 500).

In the FIG. 5, (590) shows the “egg crate” or square-array type of light cover that would typically be used with the lighting fixture so air can flow in or out. In typical industry practice, this is often made of plastic and often chromed. The size of the openings of the cover is not important to the invention.

### Detailed Description—First Alternative Embodiment

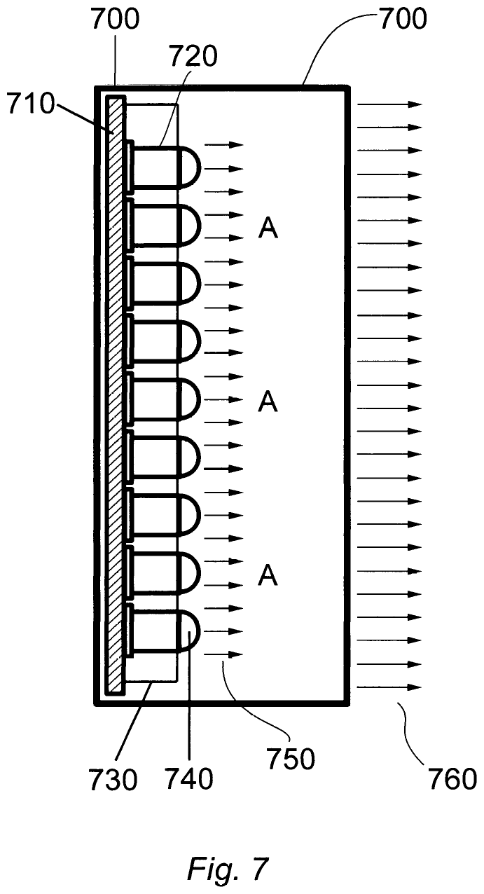

FIG. 7 is a graphical representation of an alternative embodiment of the method and invention in that the LEDs are typical 5 mm T-1¾ LED Lamps, instead of flat LED chips, wafers, or dies.

There are some potential applications wherein having a lens on each LED (720) to intensify and focus the UV-C light is an advantage. In this embodiment Printed Circuit Board (710) has a multiplicity of plastic encapsulated LEDs with integral lenses (720) mounted to it. For purposes of limiting dust accumulation, and optional encapsulation of the LED bases (730) is shown. It is anticipated that the encapsulation material could be replaced by die-cut foam, or a punched sheet metal assembly or form. In fact, (730) is optional, and depending on application may not be needed.

The lenses (740) on each LED focuses the UV-C light so that it is more concentrated than flat LEDs would supply. This would be useful a more efficient white-light output were the target, at a greater cost to manufacture. The UV-C light rays (750) would project the UV-C light into the air chamber (A) and then onto the enclosure (700) which in this instance is extruded from plastic that is doped with a white phosphor, thus negating the need for a separate coating application.

By properly orienting this light engine, the white light rays (760) would be directed to outside of the lighting fixture, and on to light the building area.

### Detailed Description—Second Alternative Embodiment

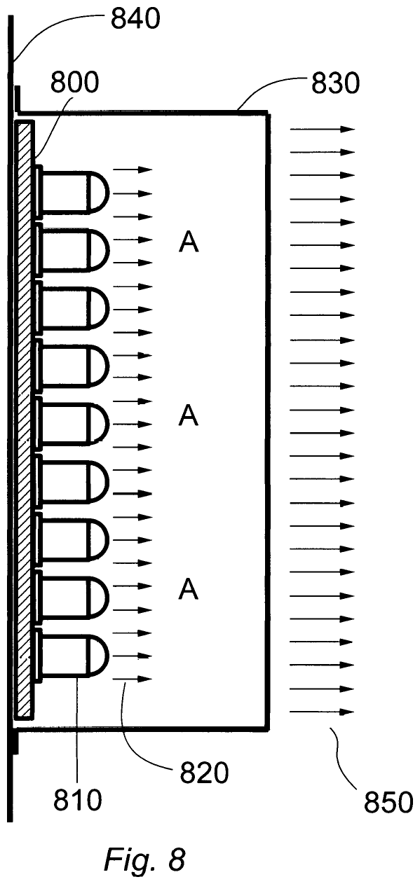

In FIG. 8, the light engine enclosure (830) is made, possibly, but not necessarily, by an extrusion process, and consists of plastic, glass, or other transparent material, doped by a white phosphor additive, thus negating the need for a separate coating process. It is anticipated that the enclosure (830) can be just of transparent material, and then have a white phosphor coating applied to the enclosure (830).

Again, we show a printed circuit board (800) that is populated with 5 mm T-1% LED lamps (810) with integral lenses. As an option, an encapsulation or die-cut plastic or foam can be placed over the LED Lamps (810) to ease the air channel (A) cleaning process. We anticipate that flat LEDs (FIG. 3, 310) can be substituted for the LED lamps (810).

The enclosure (830) is fitted over the PCB (800) and on to the base plate (840) of the lighting fixture which completes the enclosure of the LEDs.

The UV-C light rays (820) would project the UV-C light into the air chamber (A) and then onto the front of the enclosure (830), where after the white phosphor coating will cause rays of white-light (850) to be emitted.

As another variant, we anticipate that the light engine enclosure (830) can be made of formed of sheet metal or aluminum, to which an opening is made and a clear sheet with a white phosphor coating is secured over this opening. Thus, simplifying the manufacturing and assembly process, and allowing for dimensional changes without the need to purchase dies or molds.

### Detailed Description—Third Alternative Embodiment

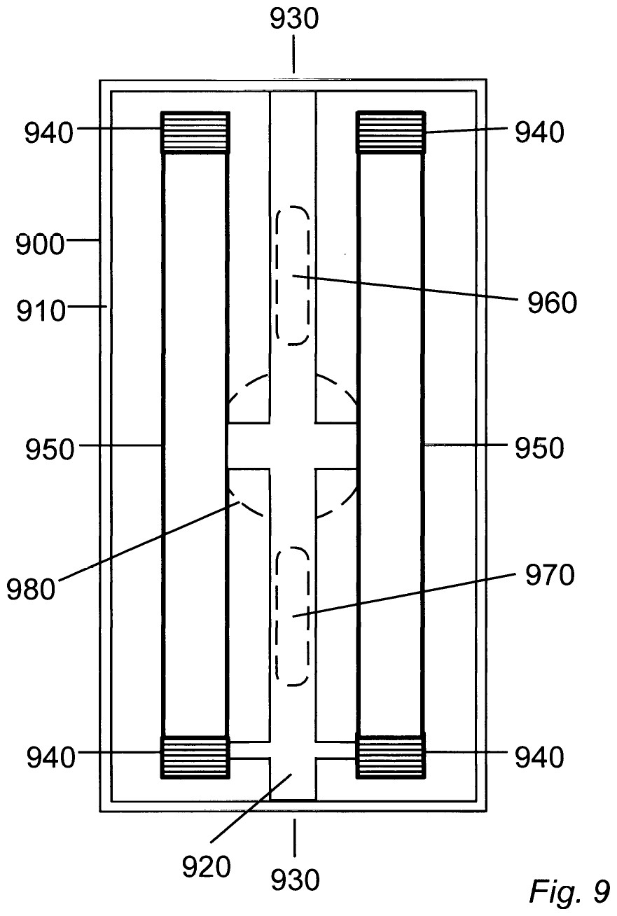

FIG. 9, is a graphical depiction of a variation of the light fixture of the first embodiment. Instead of fans (FIG. 5, 540), the lighting fixture (900) is connected to the output of the building's Heating, Ventilation, and Aid Conditioning (HVAC) system via duct (980).

Although the duct connection (980) is shown in dashed line, the channeling or ducting of that air to each light engine is not shown for reasons of simplification of the graphic. Also, for this figure the light engine assembly (light engine, channel, and vents, as one assembly) are designated as (950), with the vents (940) designated as individual subassemblies of light engine (950) for purposes of this discussion.

The air from the HVAC system is fed to the center of each of the light engine assemblies (950), wherein the untreated air is forced in both directions through the light engine air chamber. The air is disinfected under exposure of the UV-C in the interior of the light engine assemblies (950). Finally, the treated air is vented out at the vents (940) that exist at each end of the light engine assemblies (950).

Power supplies (960) and (970) power the LEDs. The connections for fan power that are in the power supplies (960) and (970) are left unconnected.

As in the light fixture of FIG. 5, the building electrical power enters at the side-mounted conduit connectors (930), and there is a frame (910) that accommodates the lighting fixture being placed in a typical 2-foot by 4-foot gridded drop ceiling.

For clarity of the graphic and discussion, the light fixture cover (FIG. 5, 590) is not shown.

### Conclusion, Ramifications, and Scope

### Conclusion

We have disclosed a method whereby air can be disinfected in lighting fixtures without significantly increasing the cost of operation over that of general lighting fixture in a building. We have discussed that UV-C light is both effective and efficient as a method for disinfecting air, and that said method is safe and results in no toxic byproducts.

One of the primary objectives of our method is to teach that in LED lighting fixtures, by not employing white LED lamps with integral white phosphor coatings as is typical in the industry, LEDs can be used to add the function of disinfecting air without significantly increasing electrical loading of the fixture. The method teaches that instead of using integral white phosphor LEDs, blue or UV LEDs can be used to generate germicidal light, and that light can be used to disinfect air that is proximate to the LEDs. The method further teaches that by employing a separate white phosphor sheet or target, the blue or UV light can then be further employed to excite phosphors so white-light can be used illuminate an area of a building. In summary, the method teaches that by separating blue or UV LED semiconductors from their white phosphor targets, an air gap or air channel can be created useful for disinfecting bioaerosols and other occurring airborne compounds in air.

Compared to other UVGI methods, our method allows for the application of UV-C to accomplish Ultra-Violet Germicidal Irradiation (UVGI) without a significant increase in operating or installation costs, as other UVGI methods require.

Although general building spaces are a prime area of application, we have revealed that the method can be modified in such a way that—with the appropriate change in phosphors—the method can be applied to agricultural lighting fixtures for the indoor growing of plants, again without a significant increase in operating costs of the light fixtures. Additionally, our method can be applied to residential areas, especially by those residents that have increased sensitivity to allergens and bioaerosols, or have weakened immune systems, or both in combination. Furthermore, and argument can be made for application of our method in pesticide storage facilities, and with explosion proof modifications, solvent storage rooms; both to lower exposure of workers to toxic byproducts of storage of these goods.

We have demonstrated variants of possible light engines useful to employ the method. We acknowledge that there are other variations of the embodiments of the light engines that can be foreseen and thus anticipated, but we have not reduced these variants to drawings.

### Ramifications

Our method provides for both air disinfection, and white light generation, without significantly increasing the cost of operations of the lighting alone.

Our method does not require the user to hide or install bulky and expensive HEPA filters or other filtering technology.

Our method does require the building operator to install and service sensors needed to monitor room occupancy to protect humans and animals from UV exposure.

With our method, UV light is not emitted outside of the lighting fixture.

Our method, employing close proximity of the air to the UV-C source, results in the air receiving high intensity UV-C exposure, effective against a broad range of microorganisms.

Our method is a chemical-free process that adds nothing to the air or environment other than white light. No applications of chemicals are needed to effect or aid in disinfection.

Unlike other methods of air disinfection, our method uses only UV-C for the disinfection of air, and hence does not require the transportation or storage of corrosive or toxic chemicals.

Our method does not create any carcinogen by-products that could adversely affect air quality or long-term human health.

As a result of the air being exposed to high-intensity UV-C energy in our method, we provide decontamination via oxygenation of several airborne solvents and pesticides.

Our method also provides decontamination of many microbial agents used as biological weapons.

With our method, there is no need to duct HVAC air flows to the lighting fixtures, however this is an option should it be desired.

Operating costs of using our method are limited to the costs of periodic cleaning, and the cost of electrical energy needed to operate the lighting fixtures.

### Scope

Although we have shown forced-air driven variants of the lighting fixtures, we anticipate that variants that depend on the existing movements of air within a building can be used to disinfect air, albeit at a lesser pace than the forced-air variants. In a similar fashion, we anticipate designs that employ our method and that of creating thermal air currents, to accomplish a more passive, and less noisy disinfection. Indeed, it is believed that even high-voltage static-charge methods can be applied to move air in application of our method without using a fan with moving parts, and gain particle removal from the air as a byproduct.

Although we have emphasized the employment of UV-C light centered at 254 nanometers, we anticipate that blue light at wavelengths up to 405 nanometers or more, even though not as efficient at disinfection, may still be useful, especially for exciting certain phosphor compounds, and can be employed in the place of UV-C light in our embodiments. The same can be said for UV wavelengths shorter than 254 nanometers.

Although the embodiments have shown light fixtures that are suitable for use in drop ceilings, we point out that our method can be applied to any light fixture of any kind. There is no intention to limit the kind of lighting fixture that can use our method, nor to limit our method to ceiling-mounted light fixtures only.

We have demonstrated our method through the employment of light engines. Our method does not depend on the employment of an assembly such as the light engines shown in illustration and discussion. To employ our method, only three things are necessary (as revealed in claim 1): 1) A source of blue or UV light; 2) A separate target of white or other color phosphor; and 3) An air gap between the two. Thus, 1 our method and invention does not necessitate manufacturing nor creating light engine assemblies.

We also anticipate that such lighting fixtures may or may not employ a grating as a visual or decorative cover in said lighting fixture. By means of example, but not by limitation, we anticipate that some variations of lighting fixtures will employ a plastic grating integrated into a swing-down door that otherwise covers the fixture so that the internal devices are not as visible, the emitted white light is more directional and thereby limiting glare from the fixture, and still provide for the movement of air in and out of the fixture.

Although the electronic power supplies as discussed herein do not incorporate self-measuring technology, we anticipate that lighting fixtures employing our method can be equipped with circuitry that can monitor the functioning of the light fixture and its components, store operational data over time, and determine the condition of any filters incorporated within the fixture. We further anticipate that said status data and operational data can be downloaded or accessed using wired or wireless communication methods.

We further anticipate that the blue or UV emitting control circuitry, can be modified so as to allow for the modulation of the blue or UV emissions, and thereby modulate the output of the white light. As revealed in U.S. Pat. No. 7,352,972B2, Method and Apparatus for the Zonal Transmission of Data Using Building Lighting Fixtures, Philip G. Franklin Sole Inventor, 2008 Apr. 1; we anticipate that such modulation can be Amplitude Modulation (AM), Frequency Modulation (FM), Time-Division Multiple-Access (TDMA) Modulation, Frequency Shift Keying (FSK), Phase Shift Keying (PSK), Pulse Code Modulation (PCM), and any of the M-ary Modulation methods, including MFSK-Orthogonal modulation. The modulated light can be used to help the blind navigate through a facility, provide data to users based upon their location, and control in-building communications. The modulation methods herein are for purposes of discussion only, and should not be construed to limit the scope of our invention.

We anticipate a variant of the lighting fixture and power supplies such that the light fixture can operate on battery power as needed.

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Part or block names as used herein are descriptive only, and should not be taken as limiting their function or purpose. Functional blocks in the figures are shown for purposes of discussion only, and nothing therein should be construed to limit their functions or circuitry, limit the number or type of outputs, imply their necessary configuration, or even imply that their presence is necessary for our invention to work. Some blocks list voltages, and discussions of voltages or currents can occur in their descriptions; the listing or discussions of voltages and currents are not meant to be limiting the scope or specification of the electronic or support circuitry herein. The inventors anticipate interfacing the method to voltages, loads, and power sources, beyond the range of those discussed or listed herein.

In this specification, words importing the singular number only, include the plural and vice versa; and words importing the masculine gender, include the feminine gender and neuter gender, and vice versa; and words importing persons include any individual, group of individuals, partnership, corporation or any business entity whatsoever; and words importing the exclusive-or shall include the inclusive-or and vice versa; and the words “section”, “subsection”, and “paragraph”, if used, and even if followed with further identification, should not be judged as limiting to any one or more provisions or clauses of this patent application.

Titles, fonts, headings of articles, sections, subsections, paragraphs, or subdivisions, are for convenience only, and neither limit nor amplify the provisions of the patent application itself. Any and all fonts and text effects used, including without limitation bolded text, underlined text, or italicized text; are for convenience only, and neither limit nor amplify the provisions of the patent application itself.

### Aid to Patent Specification—Terms Defined

“Aerobiology” is the study of microorganisms in the air that may be detrimental to human, plant, and animal health.

“Bioaerosols” are natural or artificial particles of biological origin suspended in the air, such as bacteria, viruses, mold, mildew, fungi, rickettsia, protozoa, and such microbiological byproducts such as endotoxins, mycotoxins, and Microbial Volatile Organic Compounds (“MVOCs” or “mVOCs” . . . small molecular mass substances released by microorganisms, often small molecules (<300 Da) that exhibit high-vapor pressures and low boiling points).

“Light” as used herein refers to any electromagnetic or photonic emission or ray within the range of 10 to 1050 nanometers, whether visible or not to humans. Light may also be identified as Photonic Emissions, or Photonic Energy.

Specifying Light Color

“Wavelength” is chosen in preference to “Frequency” which is wavelength's inversion (Frequency=1/Wavelength). Wavelength is scaled for convenience in nanometers [10⁻⁹ meters, or billionths of a meter], and is directly related to “the color of the light” as perceived by humans. The following table—color verses wavelength—is presented for convenience, discussion, and illustration only, and is not intended to limit any claim, process, or method.

| Color | Wavelength |
|---|---|
| Far Infra-Red | 900-1050 nm |
| Near Infra-Red | 740-900 nm |
| Red | 625-740 nm |
| Orange | 590-625 nm |
| Yellow | 565-590 nm |
| Green | 495-565 nm |
| Blue | 450-495 nm |
| Violet | 400-450 nm |
| Ultra-Violet A [Long-Wave UV] (UV-A) | 315-400 nm |
| Ultra-Violet B [Medium-Wave UV] (UV-B) | 280-315 nm |
| Ultra-Violet C [Short-Wave UV] (UV-C) | |
| (Note¹: Researchers Today Consider “Practical UV-C” | 100-280 nm |
| to start At 200 nm, and extend to 300 nm) | |
| (This is considered as the Target Range of Light | (¹200-300 nm) |
| Wavelengths that are the Wavelengths having the | |
| most effective germicidal activity) | |
| Near-UV (NUV) | 300-400 nm |
| Middle-UV (MUV) | 200-300 nm |
| Far-UV (FUV) | 100-200 nm |
| Vacuum-UV (VUV) | 10-200 nm |
| Extreme-UV (EUV) | 10-121 nm |

“UV-C” and “Germicidal UV”, from a technical standpoint, it is accepted to include within the definition of effective UV-C light to include the broader range of wavelengths between 100 nm and 300 nm. However, wavelengths below 200 nanometers are quickly and easily absorbed by air molecules, thus are in the designated spectrum of Vacuum-UV as well, and as a consequence of absorption (thus attenuation) by air molecules, are not of interest in the application of UV to disinfect air, to purify water, or to sterilize surface areas. Hence, the industrial, academic, and governmental agencies agree that, from a practical standpoint, when discussing sterilization and disinfection of microorganisms by UV exposure, UV-C should be interpreted to refer to the functionally germicidal sub-set of the spectrum of wavelengths between 200-300 nm.

Therefore, UV-C as used herein, refers to the spectrum of 200-300 nm (also designated in the chart above as “Middle-UV”). For the purposes of disinfection of microorganisms, the wavelength 254 nm is known to be most damaging and pathological to most microorganisms.

“Ray”, “Light Ray”, and “Light Rays”, are used herein to refer to a single photon, or a multiplicity of photons, in transit from a source, and not intended to be limiting in scope.

“LED” (“Light Emitting Diode”) and “LED Lamp” is as a general class of semi-conducting devices and packaged semiconductor assemblies that emit light. The terms LED and LED Lamp are used herein in the broadest sense. As a means of illustration, and without limitation, LED is intended to include: “OLEDs” (Organic Light Emitting Diodes) in the form of OLD sheets, arrays, or as individual devices; Quantum-Well Emitter LEDs as devices or as arrays; LEDs that employ lasing techniques, light emitters that use nanoscale resonating techniques, and LEDs that employ Quantum Dot techniques (“Q-dot LEDs”). Likewise, “LED” and “LED Lamp” is intended to include individual devices, arrays of dies, chips, or devices, as well as single-die or device assemblies, flexible or non-flexible sheets (such as in OLEDs underdevelopment), or flexible printed circuit assemblies. Additionally, “LED Lamp” is also intended to mean an assembly that goes beyond a single light emitting semiconductor die, which may include one or more of additional semiconductor dies, internal control or connection circuitry, internal electronic devices such as rectifiers, resistors, current-limiting or voltage-limiting devices, wire conductors or solder contacts, packaging, phosphors, and lensing.

“Sterilization”, “Deactivation”, “Inactivation” and “Disinfection” and variations thereof, when used herein and applied to bioaerosols, microorganisms, and their byproducts, are intended to convey that colonies of microorganisms are—to a significant degree—killed or damaged, so as to reduce or inhibit repair, replication, or infection. It is not intended that such terms mean 100% of all microorganisms of a targeted species are killed or lethally damaged. As a practical matter, the scientific and medical community recognize that such terms are to be interpreted to mean a significant percentage, such as 99.99% of a colony of microorganisms, are killed or inactivated. It is accepted that killing or inactivation of 100% of colonies of hundreds of thousands, if not millions, of microorganisms is difficult and often impractical.

According to Luminus Devices (Application Note APN-002805, “Ultraviolet Light for Disinfection and Sterilization”, Luminus Devices, Inc., Sunnyvale, Calif.); The degree to which a colony is impacted is the Sterility Assurance Level (SAL). In the laboratory setting, this is a logarithmic measurement and may be reported as 1 Log to 6 Log. The common representation of these measurements is:

TABLE 1
Sterility Assurance Level

| | Number of Germs | |
|---|---|---|
| SAL | Remaining out of 1 Million | Germicidal Effectiveness |

| | | | |
|---|---|---|---|
| 1 Log | 100,000 | 90% | Reduction |
| 2 Log | 10,000 | 99% | Reduction |
| 3 Log | 1,000 | 99.9% | Reduction |
| 4 Log | 100 | 99.99% | Reduction |
| 5 Log | 10 | 99.999% | Reduction |
| 6 Log | 1 | 99.9999% | Reduction |

“SAL” or “Sterility Assurance Level” as a percent effectiveness as shown in Table 1. The typical example is for a colony of one million germs. As an example of typical usage of the SAL, it is often found published on the packaging of household cleaning and disinfecting solutions, including disinfecting kitchen wipes and spray products. A 6 Log, or 99.9999% elimination of germs, is generally considered to be the level required for sterilization in a high-risk medical facility.

For non-medical, and general public atmospheres, such as households and businesses (including schools and other public buildings and areas), the control of microorganisms is targeted to 3 Log (with a 99.9% of disinfection results), or a more-preferred 4 Log (with a 99.99% disinfection reduction range). For example, the “Clorox” branded “Disinfecting Wipes” for industry and households, has a SAL of Log 3 or as stated on the package “Kills 99.9% of Viruses and Bacteria”.

The embodiments of the invention anticipated herein are to expected to provide a Sterility Assurance Level at 3 Log or higher, but in fact may be adjusted (via lighting levels or air flow rates) to provide any of the Sterility Assurance Levels from 1 Log to 6 Log.

“UVGI” refers to “Ultra-Violet Germicidal Irradiation”. UVGI is a recognized disinfection method that uses UV-C light to kill or deactivate microorganisms by destroying their nucleic acids and thereby damaging their DNA and RNA. UVGI is used in water systems to purify water, in buildings to sterilize surfaces, in labs to sterilize equipment, and in specialized HVAC (Heating, Ventilation, and Air Conditioning) systems in combination with HEPA (High-Efficiency Particulate Air) filters to disinfect room air of bioaerosols and agents of bioterrorism attack.

We claim:

1. A method for disinfecting and purifying air, comprising:
a. Means for generating ultraviolet or near ultraviolet emissions;
b. Means of an air channel wherein air is exposed to the emissions of the ultraviolet or near ultraviolet emissions; and
c. Means of a phosphor sheet or coating, said ultraviolet or near ultraviolet emissions providing excitation of phosphor means, to emit light a different color or spectrum of colors.

2. The method of claim 1, wherein the method is used within a lighting fixture.

3. The method in claim 1, wherein the emissions are ultraviolet emissions centered at 254 nanometers.

4. The method in claim 1, wherein disinfection of the air includes disinfecting bioaerosols, including one or more of bacteria, viruses, mold, mildew, fungi, rickettsia, protozoa, and such microbiological byproducts such as endotoxins, mycotoxins, and agents of biological warfare.

5. The method in claim 1, wherein purification of the air includes purification of aerosolized chemicals, including one or more of solvents or the fumes thereof, residual monomers, pesticides, and odor causing compounds.

6. The method in claim 1, wherein the source of blue or ultra violet emissions are light emitting diodes, organic light emitting diodes or films, laser diodes, or quantum well diodes, or the derivatives thereof.

7. The method of claim 1, wherein the phosphor compound used in the phosphor means is compounded for light useful for an agricultural application, including the indoor growing of plants.

8. The method of claim 1, wherein the light emissions from the phosphor means are a multiplicity of colors.

9. The method of claim 1, wherein the method is used within a lighting fixture.

10. The method of claim 1, wherein the air in the method is provided by one or more blowers or fans.

11. The method of claim 1, wherein the air in the method is provided by a building's heating, ventilation, or air conditioning system.

12. A method for disinfecting and purifying air, comprising:
Means of an enclosure, containing one or more means of a sub-assembly, with each sub-assembly means containing:
a. Means for generating ultraviolet or near ultraviolet emissions;
b. Means of an air channel wherein air is exposed to the emissions of the ultraviolet or near ultraviolet emissions; and
c. Means of a phosphor sheet or coating, said ultraviolet or near ultraviolet emissions providing excitation of phosphor means, to emit light a different color or spectrum of colors; and
d. Means to allow the white light to exit the sub-assembly enclosure.

13. The method in claim 12, wherein the emissions are ultraviolet emissions centered at 254 nanometers.

14. The method in claim 12, wherein disinfection of the air includes disinfecting bioaerosols, including one or more of bacteria, viruses, mold, mildew, fungi, rickettsia, protozoa, and such microbiological byproducts such as endotoxins, mycotoxins, and agents of biological warfare.

15. The method in claim 12, wherein purification of the air includes purification of aerosolized chemicals, including one or more of solvents or the fumes thereof, residual monomers, pesticides, and odor causing compounds.

16. The method in claim 12, wherein the source of blue or ultra violet emissions are light emitting diodes, organic light emitting diodes or films, laser diodes, or quantum well diodes, or the derivatives thereof.

17. The method of claim 12, wherein the phosphor compound used in the phosphor means is compounded for light useful for an agricultural application, including the indoor growing of plants.

18. The method of claim 12, wherein the light emissions from the phosphor means are a multiplicity of colors.

19. The method of claim 12, wherein the method is used within a lighting fixture.

20. The method of claim 12, wherein the air in the method is provided by one or more blowers or fans, or a building's heating, ventilation, or air conditioning system.
